(19) United States
(12) Patent Application Publication
(10) Pub. No.: US 2015/0290024 A1
(43) Pub. Date: Oct. 15, 2015

(54) Reusable ostomy pouch value

(71) Applicant: Achilles Nassopoulos, Montreal (CA)

(72) Inventor: Achilles Nassopoulos, Montreal (CA)

(21) Appl. No.: 13/999,975

(22) Filed: Apr. 14, 2014

(51) Int. Cl. A61F 5/445; A61F 5/441; A61F 5/44

(52) U.S. Cl. CPC A61F 5/445; A61F 5/4405; A61F 5/441; A61F 2005/4415

(57) ABSTRACT

An apparatus and system that is reusable for eliminating intestinal effluent gases and liquids from ostomy bags. The apparatus comprises an impermeable, hollow tube (1) with open extremities that is pushed half way through an orifice punctured on the upper top (10) section of an ostomy bag wall when used as a vent (5) or at the lower bottom (13) section of an ostomy bag wall when installed as a drain (6), an o-ring (21) having the hollow tube (1) section located in the interior of the inner impermeable bag wall (4) passing there through, an identical o-ring (2) having the hollow tube (1) section located at the exterior (15) outer protective bag wall (20), passing there through. The two o-rings (2) and (21) are dimensioned to fit and hold by friction or other methods around the outer circumference of the hollow tube (1), wherein the two o-rings (2) and (21) are secured face to face to adjacent regions of the interior (16) and exterior (15) protective outer bag wall (20) if existent and inner impermeable bag wall (4). A stopper 3) dimensioned to fit inside the section of the hollow tube(1) located at the exterior (15) of the bag walls (4) and (20) is inserted or removed to control the evacuation of the intestinal effluent gases and liquids.

### CROSS-REFERENCE TO RELATED APPLICATIONS

| Document Number | | | |
|---|---|---|---|
| Country Code- | | | |
| Number-Kind | Date | | |
| Code | MM-YYYY | Name | Classification |
| A] U.s.-2,667,167 | 01-1954 | P. A. Raiche | 604/339 |
| B] U.S.-3,759,260 | 09-1973 | Nolan, et al. | 604/333 |
| C] U.S.-3,865,109 | 02-1975 | Elmore, et al. | 604/339 |
| D] U.S.-4,300,560 | 11-1981 | Steer, et al. | 604/335 |
| E] U.S.-4,449,970 | 05-1984 | Bevan, et al. | 604/333 |
| F] U.S.-5,690,623 | 11-1997 | Lenz et al. | 604/333 |
| G] U.S.-5,840,073 | 11-1998 | Olsen | 604/333 |
| H] U.S.- 7,160,275 | 01-2007 | Falconer | 604/333 |
| I] U.S.-7,214,217 B2 | 05-2007 | Pedersen et al. | 604/333 |
| J] U.S.-7,604,622 | 10-2009 | Pedersen et al. | 604/333 |

### BACKGROUND OF THE INVENTION

Colostomy is the surgical procedure of making an opening from the colon to the outside of the abdomen. In a colostomy, the surgeon may remove a segment of the colon, resulting in the colon being split into two separate parts. One end of the colon is passed through a small hole in the abdominal wall. This section of intestine called a stoma, allows for body waste to leave the body from the colon. The other end of the colon, which is attached to the rectum, may either be removed or closed off with sutures and left in the abdomen. There are other similar stoma operations such as ileostomy which is a stoma from the small intestine, and urostomy which is a stoma from the urinary system. Operations are performed for temporary or permanent stomas.

Such operations are performed on humans and animals as well. In all cases, stoma wafers are used which are adhesive skin barriers or skin flanges with an orifice at the center to allow the stoma to protrude. These stoma wafers consist of either a one piece wafer and bag or a two piece wafer with a snap-on or similar attachment for the bag. The bags are also known as pouches in the ostomy field. The bags snap on and serve to hold the body waste. Many pouches are equipped with vents and filters. Still many more products are being produced specifically for stomas. Conventional methods of stoma flanges or stoma wafers or stoma skin barriers and stoma pouches allow the body wastes to leave the stoma and to fall directly into the collecting pouch. Several patents have been applied for apparatus and methods of eliminating excess liquids or venting gases and filtering odors of pouches. A stoma releases gases and liquids which accumulate in the pouch and may force the pouch to open or burst at the mechanical detachable seam unless the gas or liquid is evacuated as it builds up. Initially a valve was installed to collect gases and to filter with activated carbon the odours and send the gases to a vent bag, such is the case with

A] January 1954, the U.S. Pat. No. 2,667,167 in January 1954 by P.A. Raiche.

Later on, when ostomy bags became common several ostomy pouch venting mechanisms on ostomy pouches were patented in the U.S.

B] Sep. 18, 1973 the U.S. Pat. No. 3,759,260 was made public for a built-in vent and filter on a bag or pouch.

C] Feb. 11, 1975 the U.S. Pat. No. 3,865,109 was made public for a built in vent with a slot for venting purposes.

D] Nov. 17, 1981 the U.S. Pat. No. 4,300,560 was made public for an ostomy bag having a built in bottom drain valve on the bag held in position by a plastic block welded on the pouch.

E] May 22, 1984 the U.S. Pat. No. 4,449,970 was made public for a built in a vent and filter on a bag with a flip cap to open or shut the vent.

F] Apr. 26, 1994 the U.S. Pat. No. 5,690,623 was made public for a built in vent on a bag with a filter and an outlet opening for the filtered gases.

G] Nov. 24, 1998 the U.S. Pat. No. 5,840,073 was made public for a different style of built in vent on an ostomy bag with an original removable filter insert.

H] Jan. 9, 2007 the U.S. Pat. No. 7,160,275 B2 was made public for a complex built in vent and filter on an ostomy bag.

I] May 8, 2007, the U.S. Pat. No. 7,214,217 B2 was made public for a built in gas filter with an outlet gas aperture

J] Oct. 20, 2009, the U.S. Pat. No. 7,604,622 was made public for yet another built in vent with filter on a bag.

All of the vents, or valves, or gas release devices for ostomy bags that have been patented to date in the U.S. are built in on the bags. The valves do not come separately from the bags.

There are vents that come separately from the bag that are glued on ostomy bags for a one time use but they have not been patented in the U.S. This vent equipped with a flip up cap is glued to the upper front section of the ostomy bag. The flip up cap is opened and a hole is punctured through the center of the glued on vent. The hole punctures the center of the vent and the ostomy bag, simultaneously, allowing the gases to escape. The flip cap is kept shut to seal in the gases. The vent and flip cap resemble the vent apparatus of the U.S. Pat. No. 4,449,970, but in a format that has an adhesive back.

The present invention is for a valve used as a vent or a drain, made from rigid or flexible impermeable material such as polyethylene, wherein this valve controls ostomy pouch gas or liquid output. This valve is attached mechanically to ostomy bags is removed mechanically from the used bag before disposal of the ostomy bag, is washed and is used again on a new ostomy bag. The process can go on indefinitely since material such as polyethylene does not wear out easily in this application.

### BRIEF SUMMARY OF THE INVENTION

It is an object of the present invention to create equipment and its corresponding method of evacuating ostomy gases or liquids from ostomy pouches used on the stoma of a colostomy, ileostomy, or urostomy patient by which method the ostomy valve is produced independently from the bag. The valve is installed on the bag. A stopper is used to control mechanically the gas or liquid release. The valve is removed from the used ostomy bag, before the bag is discarded, is washed and is used again on a fresh ostomy bag.

The valve consists of
A rigid or flexible, impermeable, hollow tube that has both extremities opened.
Two identical o-rings with the same inner diameter as the outer diameter of the tube.
One rigid or flexible stopper that blocks the exhaust end of the valve

The description of the apparatus and the method of installation are as follows:

A hole smaller or equal to the diameter of the tube is punctured through the wall or walls of the ostomy bag.

The tube is pushed half way into the interior of the bag via the puncture.

One o-ring is installed onto the tube by sliding over the outer circumference of the section of the tube located in the interior of the bag.

The second-identical o-ring is installed the same way as the first o-ring onto the tube section located on the exterior of the bag.

The two o-rings are squeezed together.

The two o-rings remain in position by friction thus squeezing the bag walls without allowing gases or liquids to escape.

The two o-rings could remain in position by friction with the addition of adhesive if the unions become loose.

The two o-rings could be bolts instead of o-rings that bolt onto the tube that is threaded on the exterior.

The two o-rings could be two rings that fit together by having one ring male connecting and the other ring female connecting together or vise versa with the impermeable wall or walls of an ostomy bag fitted and held together mechanically between the snap-on rings snapped together.

The stopper is inserted inside the open exhaust end of the valve tube at the outside of the bag.

The stopper is removed and put back as required to control gases or liquids.

Before the used ostomy bag is discarded, the o-rings are detached, the tube is removed from the ostomy bag and all the items, including the stopper are washed and used again.

The valve is used indefinitely on new or undamaged ostomy bags.

With this invention:
1. Pouch gases will be released without accidents from intestinal effluent which happens when pulling even slightly apart the ostomy pouch mechanically connecting ring of the ostomy pouch and wafer to release gas pressure from pouches not equipped with vents.
2. Pouch valves will be more environmentally friendly because they will be recycled indefinitely.
3. It will be more economical to control ostomy bag gases not having to purchase vents that are for a one time use only, such as the vent types that are glued on the bag. This type of vent cannot be unglued from the used ostomy bag and used again on a new bag.
4. It will be more economical to control ostomy bag gases or liquids not having to purchase ostomy bags with built in valves. Ostomy bags with built in valves as vents or drains are more costly than bags without.
5. Proper venting will increase the duration of wafers on the skin by preventing the premature lifting of the wafer off the skin due to excessive ostomy gas pressure.
6. Proper venting will reduce leaks around a stoma wafer orifice and therefore reduce infections from leaks because the wafer stays in place longer due to less ostomy gas pressure as explained in item No. 5 above (of this “With this invention” list).
7. The ostomy pouch valve will be a universal item that could be used on all ostomy bags that exist and it will be an item that will never wear out nor go out of style as long as ostomy pouches will exist.
8. The valve can be installed on pouches with various layers of material penetrating through more than one wall of bag. For instance the vent will work properly on an inner impermeable bag that is covered with a protective outer cloth bag.
9. The valve can be installed on one piece ostomy pouch and wafer assemblies.

### BRIEF DESCRIPTION OF THE SEVERAL VIEWS OF THE DRAWING

FIG. 1 is the side view of the ostomy appliance valve parts shown not assembled.

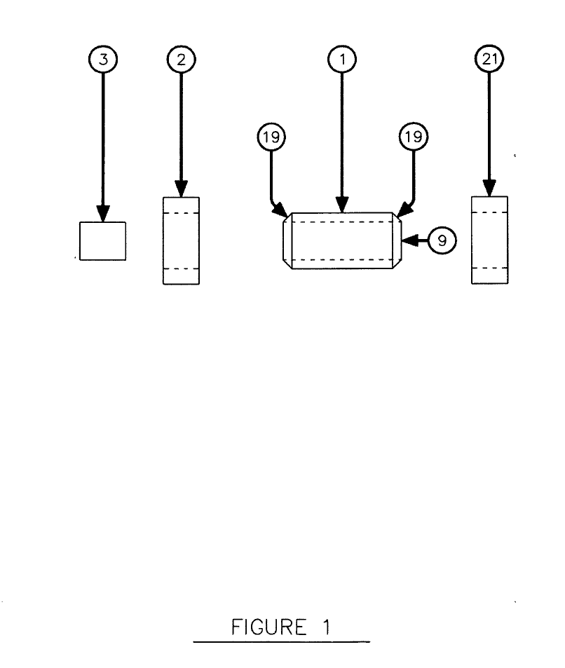

FIG. 2 is the ostomy appliance valve shown three (3) dimensionally assembled on the bag walls

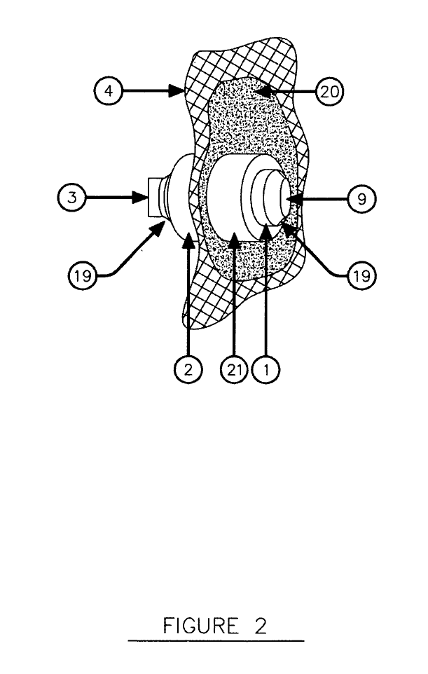

FIG. 3 is the side view of the ostomy appliance valve parts assembled on the bag walls.

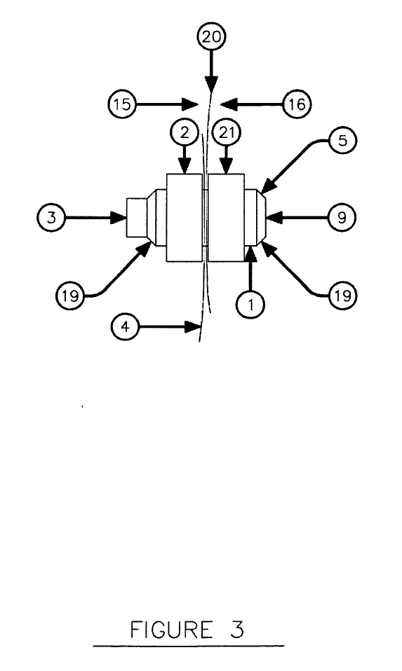

FIG. 4 is the ostomy appliance valve assembled as a vent.

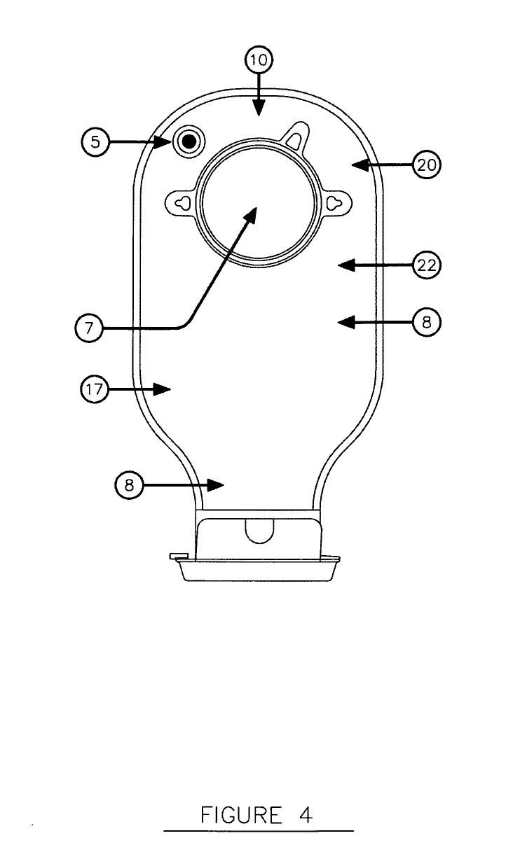

FIG. 5 is the ostomy appliance valve assembled as a drain.

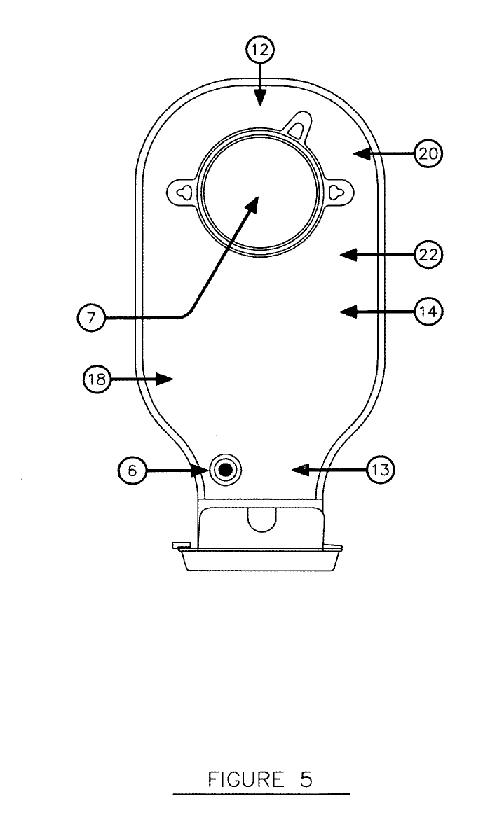

### DRAWINGS DETAILED EMBODIMENT

1) Tube

2) Exterior o-ring

3) Stopper

4) Inner impermeable ostomy bag wall

5) Vent

6) Drain

7) Pouch stoma opening

8) Front

9) Hollow

10) Top

11) Bottom

12) Top

13) Bottom

14) Front

15) Exterior

16) Interior

17) Exterior

18) Exterior

19) Tapered

20) Outer protective ostomy bag wall

21) Interior o-ring

22) Ostomy bag

### DETAILED DESCRIPTION OF THE INVENTION

The present invention is the method and the equipment invented to eliminate or evacuate stoma gases and liquids.

The valve consists of the following parts:

One (1) tube that:

Is a hollow cylindrical, rigid or flexible impermeable tube:

Has both open ends tapered on the outer circumference.

Is from ¼ inch to 1 inch long.

Has an internal diameter of 1/16 to ¼ inches

Has a wall thickness of 1/64 to 1/16 inches.

Two (2) identical o-rings that:

Are rigid or flexible.

Are impermeable.

Have the same internal diameter or slightly smaller than the external diameter of the tube used.

Have a wall thickness of 1/64 to ¼ inches.

Have a width of 1/64 to ¼ inches

One (1) stopper that:

Is of rigid or flexible material

Is impermeable.

Is cylindrical, square, or rectangular as long as the mass of the stopper is large enough to plug either open end of the tube.

The method of use of the ostomy pouch valve is as follows:
A) The wall of the ostomy bag or pouch is punctured. If the ostomy bag has more than one wall, as there might be a protective outer wall and an inner impermeable wall, all the walls are punctured.
B) The valve tube is forced half way through the punctured wall or walls.
C) One of the two identical o-rings is installed by sliding onto the exterior surface of the section of the tube located in the interior of the ostomy bag.
D) The other identical o-ring is installed by sliding onto the exterior surface of the section of the tube located at the exterior of the ostomy bag.
E) The two (2) identical o-rings are installed are squeezed together thus squeezing in between them the wall or walls of the bag.
F) The o-rings hold in place by friction.
G) The tight squeezing together mechanism of the o-rings and bag walls creates a sufficient seal and grip to prevent the tube from loosening or leaking.
H) If leaks develop, an adequate adhesive can seal them.
I) The stopper is inserted and wedged securely inside the exhaust open end of the tube section that is on the exterior of the bag.
J) When the used bag is discarded, the valve is recuperated and is used again on a new bag.

Please note that similar valves using two rings and a tube could be produced but will be considered as proprietary to this invention because of the use of the principle of a tube, two o-rings and a stopper.

Instead of o-rings squeezing the bag walls together the valve could be in the form of:

A hollow tube that is threaded on the outside with bolts squeezing the bag walls together when the bolts are bolted closer together with the pouch walls wedged securely between them.

A hollow tube that uses a male mechanically connecting ring on the tube in the inside of the bag and a female connecting ring on the tube on the outside of the bag or vise versa and the two male/female rings are mechanically connected together thus wedging the ostomy bag walls securely between them.

This valve could be used as a vent by installing at the upper portion of the ostomy bag or as a drain valve by installing at the lower section of the ostomy bag.

In all cases adhesive could be added to the unions for further solidification.

1. An ostomy appliance for evacuating ostomy gases or liquids from an ostomy bag comprising:
a drain valve secured in an opening created on the ostomy bag walls consisting of an outer protective ostomy bag wall if existent and an inner impermeable ostomy bag wall, said valve comprising a hollow tube that is pushed half way through said walls via said opening into the interior of said ostomy bag, an o-ring having said tube located inside of said interior impermeable ostomy bag wall passing there through, an o-ring identical to said o-ring having said tube located at the exterior of said walls passing there through, and a stopper dimensioned to fit in said tube located at the exterior of said ostomy bag walls to insert or remove from said tube to control gas or liquid release.

2. The ostomy appliance of claim 1 wherein said two identical o-rings have the same inner diameter as the outer diameter of said tube, plus or minus 1/32 of an inch

3. The ostomy appliance of claim 1 wherein both open extremities of the exterior surface of said tube are tapered.

4. The ostomy appliance of claim 1 wherein said o-ring at the interior of said bag walls and said o-ring at the exterior of said bag walls are secured face-to face to adjacent regions of the interior and exterior said bag walls.

5. The ostomy appliance of claim 1 wherein said valve is recyclable because it is reusable from one pouch to the next without damage.

6. The ostomy appliance of claim 1 wherein said valve is installed at the upper top region of said ostomy bag when used as a vent.

7. The ostomy appliance of claim 1 wherein said valve is installed at the lower bottom region of said ostomy bag when used as a drain.

8. The ostomy appliance of claim 1 wherein the items of said appliance are made from rigid or flexible material.

9. The ostomy appliance of claim 1 wherein the items of said appliance are made from impermeable material.

10. The ostomy appliance of claim 1 wherein the invention is for holding a hollow tube between ostomy bag walls by two rings with a stopper on the exhaust end of the tube outside the ostomy bag. All the apparatus used being reusable and recyclable. The claim holds regardless of the method used to secure the said valve in place because said o-ring inside said bag and said o-ring outside said bag remain in position by friction but can also stay in position by other methods and mechanisms such as the use of a hollow threaded tube and bolts, or the use of male and female mechanically connecting rings.
